(19) United States
(12) Patent Application Publication
(10) Pub. No.: US 2019/0355449 A1
(43) Pub. Date: Nov. 21, 2019

(54) BIOLOGICAL INFORMATION COLLECTION SYSTEM, INPUT DEVICE, AND BIOLOGICAL INFORMATION COLLECTION DEVICE

(71) Applicant: RENESAS ELECTRONICS CORPORATION, Tokyo (JP)

(72) Inventor: Takeshi FUKUNAGA, Tokyo (JP)

(21) Appl. No.: 16/400,897

(22) Filed: May 1, 2019

(30) Foreign Application Priority Data
May 16, 2018 (JP) 2018-094441

(51) Int. Cl. G16H 10/60; G06F 3/0482; G06F 3/0488; G06F 3/0484; G16H 50/30

(52) U.S. Cl. CPC G16H 10/60; G06F 3/0482; G16H 50/30; G06F 3/0484; G06F 3/04883

(57) ABSTRACT

The current health information and the biological information are synchronized and associated with each other. The biological information collection system includes a health information input device 1 and a biological information collection device 2. The health information input device 1 inputs health information indicating a health condition of a certain date and time and first time information indicating the certain date and time. The biological information collection device 2 acquires biological information, and collects the biological information in association with the second time information indicating the acquired date and time. The biological information collection system includes a generation unit that generates tag information based on the health information and the first time information, and includes a storage unit that stores the tag information in a state of being associated with the biological information based on the second time information and the first time information indicated by the tag information.

### CROSS-REFERENCE TO RELATED APPLICATIONS

The disclosure of Japanese Patent Application No. 2018-094441 filed on May 16, 2018 including the specification, drawings and abstract is incorporated herein by reference in its entirety.

### BACKGROUND

The present invention relates to a biological information collection system, an input device, and a biological information collection device, for example, a biological information collection system that stores biological information in a state in which health information is associated, an input device for the health information, and a biological information collection device that collects the biological information.

In Japanese Patent Laid-open Publication No. 2016-033805 there is disclosed a system for dynamically updating candidates of tags that can be assigned. In this system, tag candidates that can be assigned based on the date or the like are displayed in response to input of an image file or the like acquired in the past.

In Japanese Patent No. 5,210,471, there is disclosed a technique related to flick character input.

### SUMMARY

However, since the system described in Patent Document 1 cannot provide tags in real time, even when it is applied to a system for associating health information with biological information, there is a problem that the current health information and the biological information cannot be synchronized and associated with each other.

Other objects and novel features will become apparent from the description of this specification and the accompanying drawings.

According to one embodiment, there is provided a biological information collection system comprising: an input device for inputting health information on a health condition at a certain date and time and first time information indicating the certain date and time; a biological information collection device for acquiring biological information and collecting the biological information in association with second time information indicating the acquired date and time; a generation unit for generating tag information based on the health information and the first time information; and a storage unit for storing the tag information in association with the biological information based on the second time information and the first time information indicated by the tag information.

According to the above embodiment, the tag information generated from the current health information can be associated with the biological information in real time by synchronizing the date and time.

### BRIEF DESCRIPTION OF THE DRAWINGS

FIG. 1 is a block diagram illustrating a configuration example of a biological information collection system according to a first embodiment;

FIG. 2 is a flow chart illustrating an example of processing in a biological information collection system.

FIG. 3 is a schematic diagram for schematically explaining a state in which data is merged based on a date and time synchronized with synchronization between two devices

FIG. 4 is a schematic diagram illustrating a flow of processing in a biological information collection system;

FIG. 5 is a conceptual diagram illustrating an example of an input operation in a touch panel.

FIG. 6 is a block diagram illustrating an example of a configuration of a part for generating tag information in an input device;

FIG. 7 is a conceptual diagram illustrating a state of time synchronization between a terminal device and a wearable device.

FIG. 8 is a diagram illustrating an example of a wearable device and the biological information collected by the device.

FIG. 9 is a diagram illustrating an image of data stored in a wearable device;

FIG. 10 is a diagram illustrating an example of the correspondence between a touch portion and a living body part.

FIG. 11 is a diagram illustrating the distance between the starting point and the end point.

FIG. 12 is a diagram illustrating an example of the correspondence between the input end point coordinate and the symptom.

FIG. 13 is a diagram illustrating an example of a series of GUI images including a GUI image that prompts the input of symptom improvement.

FIG. 14 is a diagram illustrating an example of a series of GUI images for inputting symptom improvement.

FIG. 15 is a diagram illustrating an example of a series of GUI images in which the severity of symptom improvement is input.

FIG. 16 is a diagram illustrating an example of a series of GUI images in which the severity of the symptom deterioration is input.

FIG. 17 is a conceptual diagram illustrating an example of biological information associated with health information.

FIG. 18 is a conceptual diagram illustrating other examples of biological information associated with health information.

FIG. 19 is a diagram illustrating an example in which biological information is classified and stored for each piece of health information.

FIG. 20 illustrates another example of a GUI image shown in FIG. 5.

FIG. 21 illustrates another example of a GUI image shown in FIG. 5.

FIG. 22 is a flowchart illustrating an example of a processing in a biological information collection system according to the second embodiment;

FIG. 23 is a diagram illustrating an example of a series of GUI images for inputting the cause of symptoms.

FIG. 24 is a diagram illustrating an example of a series of GUI images for inputting reasons for symptom improvement.

FIG. 25 is a schematic diagram illustrating another example of an input in a touch panel.

FIG. 26 is a diagram illustrating an example of a hardware configuration included in a device in a biological information collection system.

### DETAILED DESCRIPTION

Embodiments to which means for solving the above problems is applied will be described in detail below with reference to the drawings. For clarity of explanation, the following description and drawings are appropriately omitted and simplified. In addition, the respective elements described in the drawings as functional blocks for performing various processing can be configured by a CPU (Central Processing Unit), a memory, and other circuits in terms of hardware, and are realized by programs loaded in the memory in terms of software. Therefore, it is understood by those skilled in the art that these functional blocks can be realized in various forms by hardware alone, software alone, or a combination thereof, and the present invention is not limited to any of them. In the drawings, the same elements are denoted by the same reference numerals, and a repetitive description thereof is omitted as necessary.

Also, the programs described above may be stored and provided to a computer using various types of non-transitory computer readable media. Non-transitory computer readable media includes various types of tangible storage media. Examples of non-transitory computer-readable media include magnetic recording media (e.g., flexible disks, magnetic tapes, hard disk drives), magneto-optical recording media (e.g., magneto-optical disks), CD-ROM (Read Only Memory), CD-R, CD-R/W, solid-state memories (e.g., masked ROM, PROM (Programmable ROM), EPROM (Erasable PROM, flash ROM, RAM (Random Access Memory)). The program may also be supplied to the computer by various types of transitory computer-readable media. Examples of transitory computer-readable media include electrical signals, optical signals, and electromagnetic waves. The transitory computer readable medium may provide the program to the computer via wired or wireless communication paths, such as electrical wires and optical fibers.

In the following embodiments, when it is necessary for convenience, the description will be made by dividing into a plurality of sections or embodiments, but except for the case where it is specifically specified, they are not independent of each other, and one of them is related to a modification, an application, a detailed description, a supplementary description, or the like of part or all of the other. In the following embodiments, the number of elements or the like (including the number, numerical value, quantity, range, and the like) is not limited to the specific number except the case where it is specified in particular or the case where it is obviously limited to the specific number in principle, and may be more than or equal to the specific number, or may be less than or equal to the specific number.

Furthermore, in the following embodiments, the constituent elements (including the operation steps and the like) are not necessarily essential except for the case in which they are specifically specified, the case in which they are considered to be obviously essential in principle, and the like. Similarly, in the following embodiments, when referring to a shape, a positional relationship, or the like of a component or the like, it is assumed that the shape or the like is substantially approximated or similar to the shape or the like, except for the case where it is specifically specified and the case where it is considered to be obvious in principle. The same applies to the above-mentioned numbers and the like, including the number, the numerical value, the amount, the range, and the like.

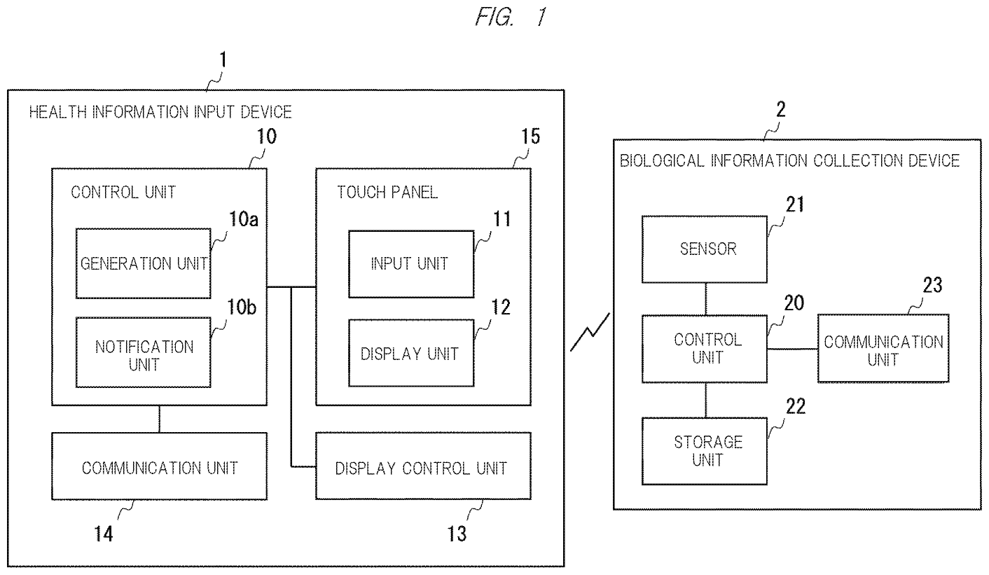

FIG. 1 is a block diagram illustrating a configuration example of a biological information collection system according to Embodiment 1. As shown in FIG. 1, the biological information collection system (hereinafter, the system) according to the present embodiment may include an input device (health information input device) 1 for inputting health information, and a biological information collection device 2 for collecting biological information.

The health information input device 1 is a device for inputting health information indicating a health condition of a certain date and time and first time information indicating the certain date and time. The health information may include, for example, information indicating emotion or behavior in addition to information indicating symptoms. The health information may also be referred to as health status information. The first time information can be acquired by, for example, a clock function provided in the health information input device 1.

The health information input device 1 can be realized by, for example, installing input programs for performing input by input methods as described later in terminal devices such as PCs (Personal Computer), mobile phones (including those called smartphones), tablet terminals, and the like. For example, the terminal device can fulfill the function of the health information input device 1 by employing a configuration in which a GUI (Graphical User Interface) image is displayed according to input procedures described later. Here, the PC may be a tablet type, a desktop type, or a notebook type.

On the other hand, the biological information collection device 2 is a device that acquires biological information and collects the biological information in association with a second time information indicating an acquired date and time. The biological information is basically periodically acquired in accordance with an acquisition operation or the like. The second time information can be acquired by, for example, a clock function provided in the biological information collection device 2.

The system includes the following generation unit and storage unit. The generating unit generates tag information based on the health information and the first time information. The tag information can be said to be information in which health information of a certain date and time and time information indicating the date and time are linked. FIG. 1 illustrates an example in which the generation unit is provided as the generation unit 10a on the health information input device 1 side. In this case, the health information input device 1 may be referred to as a tag input device or a tag information input device. Although this example will be described below, it is also possible to configure the biological information collection device 2 or another device such as a PC to acquire the health information and the first time information from the health information input device 1 and generate the tag information.

The storage unit stores the tag information in a state in which the tag information is associated with the biological information based on the second time information and the first time information indicated by the tag information. The storage unit may be a memory such as a nonvolatile memory. The biological information with which the tag information is associated can be generated by, for example, having an association unit for performing the association. FIG. 1 illustrates an example in which the storage unit 22 is provided on the side of the biological information collection device 2. Hereinafter, description will be made on the basis of this example and on the assumption that the above-mentioned association unit is included in the control unit 20. However, the health information input device 1 or another device such as a PC may be configured to have a storage unit for storing tag information in a state of being associated with biological information, or may be configured to perform the association.

The health information input device 1 and the biological information collection device 2 of the present system will be described based on the configuration example of FIG. 1. As shown in FIG. 1, the health information input device 1 may include a control unit 10 having the above-described generation unit 10a and a notification unit 10b to be described later, an input unit 11, a display unit 12, a display control unit 13, and a communication unit 14. The biological information collection device 2 may include a control unit 20, a sensor 21, the above-described storage unit 22, and a communication unit 23.

The control unit 10 of the health information input device 1 includes the generation unit 10a and the notification unit 10b, and controls the entire health information input device 1. The input unit 11 is a unit for inputting various information such as health information by a user operation. The health information input from the input unit 11 can be stored in a storage unit, not shown, in the health information input device 1.

The display unit 12 may be a display panel such as a liquid crystal panel or an organic electroluminescence panel. The display control unit 13 is a part that controls display on the display unit 12, and can be provided in the control unit 10. The input unit 11 may include a touch sensor or the like, and in this case, the input unit 11 and the display unit 12 may constitute the touch panel 15. However, the input unit 11 may be configured by hardware such as a keyboard and buttons.

The communication unit 14 can be configured by a communication adapter or the like for communicating with the communication unit 23, and any communication method may be used as long as communication with the communication unit 23 is possible. However, from the viewpoint of convenience, it is preferable that the communication unit 14 is a wireless communication unit that performs communication based on a short-range wireless communication standard such as the Bluetooth (registered trademark) standard. The same applies to the communication unit 23, which can be configured by a communication adapter or the like for performing communication with the communication unit 14, and any communication method may be used as long as communication with the communication unit 14 is possible. The communication unit 14 and the communication unit 23 perform short-range radio communication based on the Bluetooth (registered trademark) standard in the following examples.

The control unit 20 of the biological information collection device 2 controls the entire biological information collection device 2. The sensor 21 is an example of a biological information acquiring unit for acquiring biological information, and is provided with a plurality of types depending on the type of biological information to be acquired. The biological information may also be referred to as biological sensing information. The sensor 21 can control the timing of sensing by the control unit 20, but can also periodically transfer acquired information to the control unit 20.

As described above, according to the present system, rather than inputting past health information, it is possible to input health information such as current symptoms, and it is possible to associate health information and biological information by synchronizing the date and time. Therefore, according to the present system, it is possible to associate the tag information generated from the current health information in real time with the biological information by synchronizing the date and time. Further, according to the present system, it is possible to avoid a situation in which the user does not remember the information to be input, and it is possible to store an accurate date and time of the symptoms and the like.

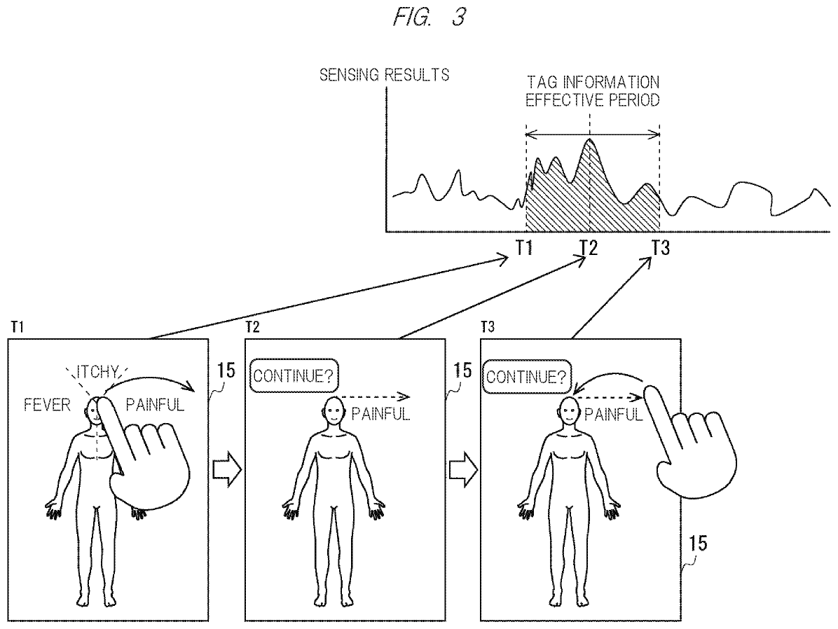

The system may also collect information necessary, for example, to create a disease model. In the present system, as the necessary information, it is possible to continuously collect various types of biological information, collect health information at each time, and store them in association with each other. Here, when creating a disease model or the like, it is very important to know which date and time of the biological information the health information corresponds to, for example, a tag information of effective period as shown in FIG. 3 to be described later. Therefore, it is necessary to synchronize and associate the information collected or input by both the biological information collection device 2 and the health information input device 1. In the present system, such synchronized association is possible by the above-described configuration. In particular, biological information can be continuously acquired along the time axis and health information can be input discretely on the time axis. According to the present system, by synchronizing and associating the biological information and the information of the effective period of the health information, it is possible to easily associate the biological information continuously along the time axis and the health information discretely input on the time axis with each other over time. According to the present system, it is possible to create a database capable of accurately extracting biological information and health information.

The present system can adopt the following various configurations in such a configuration. It is preferable that the health information input device 1 is configured to be capable of inputting, as one type of health information, progress information indicating a progress health condition. For example, the health information input device 1 may be configured to display a GUI image for inputting progress information. The progress information can be said to be information indicating the progress with respect to the previously input progress information, including the initial information which is the first input information. It should be noted that other types of user interfaces, such as a character user interface, may be employed instead of the GUI images described in this example and later.

The notification unit 10b performs notification to prompt input of the progress information based on the already input health information. The notification unit 10b can be configured to perform such notification by displaying, for example, a pop-up display of a GUI image or the like, but can also be configured to perform notification by voice or vibration. The frequency of notification may be determined in advance as every day, every half day, every meal time, or the like. The health information input device 1 may adopt a configuration that does not include the notification unit 10b, but by providing the notification unit 10b, can lead the user to input a large amount of health information.

The input unit 11 of the health information input device 1 can be configured to be capable of inputting the above-described initial information as one type of health information. Initial information refers to information indicating an initial health condition for a symptom or a body part. The symptom may be expressed, for example, “painful”, “itchy”, or the like, but may be other emotions, for example, “pleasant”, “sad”, or the like. Here, the input unit 11 can input “pleasant” as, for example, information indicating “no problem” and “sad” as, for example, information indicating “pain” and the like, respectively. As described above, the input unit 11 can be configured to be capable of inputting the progress information as one type of health information. The progress information can be said to be information indicating the progress from the initial health condition, but when there is a plurality of pieces of progress information, it can also be said to be information indicating the progress from the health condition indicated by the preceding progress information.

The health information input device 1 is configured to be able to input the first time information about the health information as described above, and hereinafter, the first time information for the initial information is referred to as initial time information, and the first time information for the progress information is referred to as progress time information.

It is preferable that the generation unit 10 a generates the tag information about the initial information based on the initial information, the initial time information, and the progress time information (progress time information about the progress information input for the symptom after the initial information). As a result, the storage unit 22 can store tag information indicating a certain symptom in association with the biological information from the date and time indicated by the initial time information to the date and time indicated by the progress time information.

When no more progress information about the same symptom is input after the initial information or the progress information, the effective period of the health information may be defined by dividing a predetermined period, for example, one hour (i.e., the last date and time of the health information).

The display control unit 13 can be configured to display the following GUI image on the display unit 12. In other words, this GUI image is a GUI image in which an area representing a body part (hereinafter referred to as a “body part area”) is displayed in a selectable state, and when a body part is selected, an area representing a symptom that may occur in the selected body part (hereinafter referred to as a “symptom area”) is displayed in a selectable state. Here, the body part area is displayed so as to be selectable for each body part, and the symptom area is also displayed so as to be selectable for each symptom.

The body part area may be, for example, an area indicating a body part's name, but it is preferable to be an area indicating a body part in the schematic diagram of the body. The schematic diagram may also be referred to as an illustration, a picture, or the like. By displaying a schematic diagram of the body and selecting the part, it is possible to make the part visually easy to understand for the user and to easily select the part. The symptom area may be an area indicating a symptom's name, or may be an area of a schematic diagram representing a symptom.

In this case, the input unit 11 inputs information representing the body part and symptoms corresponding to the body part area and symptom area selected in the GUI image as at least a part of the health information (i.e., as part or all of the health information).

Symptoms and body parts can also be entered in reverse order. In other words, the display control unit 13 may display a GUI image in a selectable state indicating a symptom area, and when the symptom area is selected, a body part area indicating a body part where a symptom may occur corresponding to the selected symptom area may be displayed in a selectable state on the display unit 12.

The display control unit 13 may be configured to display the following GUI image on the display unit 12. In other words, the GUI image is a GUI image in which a body part area is displayed in a selectable state, and when the body part area is selected, a symptom area representing a symptom that may occur in a body part corresponding to the selected body part area is displayed in a selectable state.

In this case, the input unit 11 determines the severity of the symptom in accordance with the selected position in the area or the distance from the predetermined position in the area to the selected position in the symptom area. For example, in the case of an operation using the touch panel 15, the position at which the user releases the finger by performing a sliding operation can be set as the selected position in the symptom area.

The input unit 11 inputs information representing a body part area and a symptom area corresponding to the selected body part area and information representing a severity of the symptom as at least a part of the health information. For example, in the case of an operation using the touch panel 15, information indicating the severity corresponding to the distance between which the user performed the slide operation is input.

Such an input method can be used for inputting initial information, but can also be used for inputting progress information, and for example, if there is initial information having the same body part and symptom input, the information input this time can be handled as progress information.

The input of the progress information can also be performed as follows. First, the display control unit 13 may be configured to display the following initial GUI image on the display unit 12. That is, the initial GUI image is a GUI image in which a body part area is displayed in a selectable state, and in the case where the body part area is selected, a symptom area representing a symptom that may occur in a body part area corresponding to the selected area is displayed in a selectable state.

The input unit 11 determines the severity of symptoms according to the distance from the selected position in the body part area or the predetermined position in the area (e.g., the center position) to the ultimately selected position (the selected end position) in the symptom area. In the case of the operation using the touch panel 15, the position at which the user releases the finger by performing the slide operation can be set as the selected end position.

The input unit 11 inputs information representing a body part and a symptom corresponding to the selected body part area and a symptom area in the initial GUI image, and information representing a severity which are selected in the initial GUI image as at least a part of the initial information.

Further, the display control unit 13 can be configured to display the progress input GUI image for showing the information indicating the severity in the initial information in a state in which the information can be changed (e.g., the distance can be changed) on the display unit 12. That is, the progress input GUI image can accept an operation for changing (updating) the information indicating the severity of the symptom. Then, the input unit 11 inputs the information indicating the severity of change and the information indicating the body part and the symptom as at least a part of the progress information in the progress input GUI image.

Such a change operation can be, for example, a slide operation for returning to the starting point direction from the end point of the slide operation at the time of initial information input, or a slide operation for returning to the starting point from the middle. Such a slide operation can be accepted, and the severity of the symptom can be determined and updated in accordance with the remaining amount up to the starting point or in accordance with the slide amount. Note that, even if the health information is not information in which the severity of the symptom is determined by the slide operation on the initial GUI image, information indicating the severity may be displayed on the progress input GUI image.

By employing any of the various methods of inputting health information using GUI images as described above, it is possible to reduce labor as compared with the case of inputting health information as character information for each character. For example, when a flick character input as described in Patent Document 2 is employed, it takes time and labor to input characters in order to select characters one by one. More specifically, in the case of inputting information of “headache” as health information, it is necessary to input flick characters seven times or ten times such as “I have a headache” or “I have a terrible headache”, and input becomes troublesome. Further, in the case of using such flick character input, since a free sentence is input, a problem may arise that even in the case of the same symptom, for example, “headache” and “headache” cannot be input uniquely. In addition, when such flick character input is used, not only is there a language dependency, but the quantitative expression of symptoms becomes difficult as in the case of normal character input. On the other hand, according to the input method using the GUI image as described above, these problems can be solved. The language dependency can be solved by using a schematic diagram in the GUI image.

The health information may also include information indicating the body part and symptoms as described above. It is preferable that the biological information is stored for each combination of the body part and the symptom in accordance with the health information indicated by the tag information for analysis (analysis of the relationship between the symptom and the biological information of the user) to be performed later. Of course, in this case, biological information in the same time zone may be associated with tag information for different combinations. In addition, the biological information can be stored as an individual database for each combination of symptoms and body parts, and, for example, a biological information database classified for each combination of symptoms and body parts can be created.

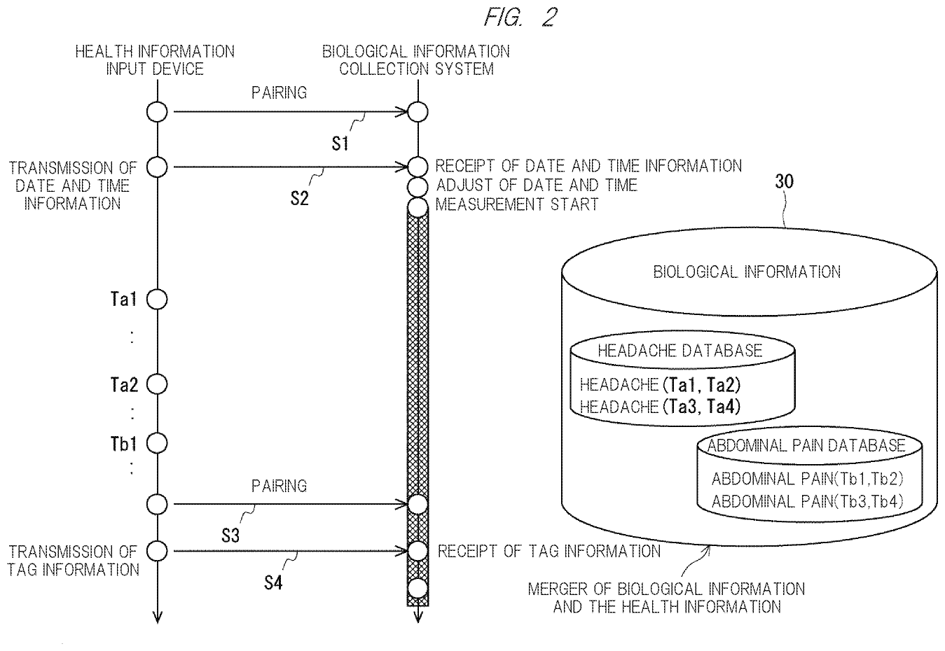

Next, an example of a specific process in the present system will be schematically described with reference to FIGS. 2 to 6. First, time synchronization in the present system will be described with reference to FIGS. 2 and 3. FIG. 2 is a flow chart illustrating an example of processing in the present system, and FIG. 3 is a schematic diagram for schematically explaining a state in which data is merged based on a date and time synchronized with synchronization between two devices.

The present system performs time synchronization (date and time synchronization) between at least two devices of the health information input device 1 (hereinafter, the input device 1) and the biological information collection device 2 (hereinafter, the collection device 2). For this purpose, in the present system, health information of a certain date and time, which may be progress information, is output from the input device 1 to the collection device 2 as tag information, and the tag information is associated with the biological information.

As shown in FIG. 2, first, pairing is performed between two devices (between the communication unit 14 and the communication unit 23) (step S1), and then, the input device 1 transmits date and time information to the collection device 2 (step S2). As a result, the date and time in the clock function of the input device 1 and the collection device 2 can be adjusted.

After step S2, the input device 1 accepts input of health information from the user, and generates one or a plurality of pieces of tag information, here, tag information Ta1, Ta2, Tb1, and the like. For the sake of convenience, in the following, for example, the first time information (input date and time information) of the tag information Ta1 is also represented as “Ta1”, and the name and date and time of the tag information are represented by the same symbol. On the other hand, in the collection device 2, the acquisition (measurement) of the biological information is started by the measurement start operation of the user or the like. The collection device 2 measures the biological information continuously at the date and time synchronized in step S2, for example, at a predetermined interval, and collects the biological information in association with the second time information. Note that the interval of measurement of the biological information can be varied depending on the type of the sensor 21.

Then, for example, by a user operation, the input device 1 performs pairing with the collection device 2 (step S3), and transmits the tag information generated from the health information to the collection device 2 (step S4). Before the tag information is transmitted, the input device 1 and the collection device 2 do not have to be connected to each other. The collection device 2 receives the tag information transmitted in step S4, and merges the biological information and the health information by associating the tag information with the biological information.

As a result, for example, tag information in which the health information indicates headache (the body part is the head and the symptom is the pain) is stored in the database 30 in association with the biological information of the period (effective period) indicated by the tag information. This period will be described later. In this example, the tag information of the effective period Ta1 to Ta2 and the tag information of the effective period Ta3 to Ta4 exist with respect to the headache, and the respective tag information is associated with the biological information of the respective effective periods. Similarly, the tag information in which the health information indicates abdominal pain is stored in the database 30 in association with the biological information in the period indicated by the tag information. With respect to abdominal pain, tag information of effective periods Tb1 to Tb2 and tag information of effective periods Tb3 to Tb4 exist, and each tag information is associated with biological information of each effective period. As shown in FIG. 2, the biological information can be stored as individual databases for each piece of health information.

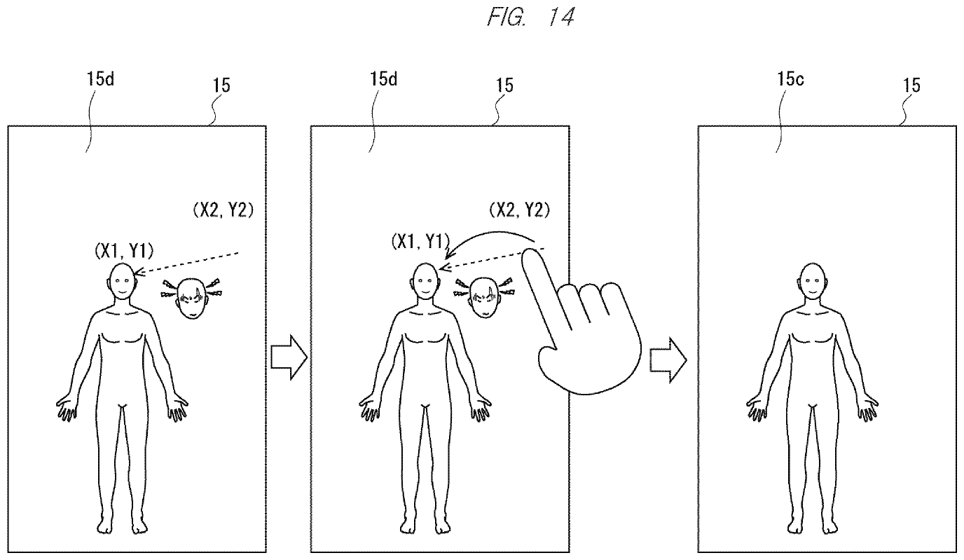

The period indicated by the tag information will be described with reference to FIG. 3. As illustrated in FIG. 3, description will be given of a case where tag information is generated based on the health information (initial information) input at the date and time T1, progress information indicating the same content is input at the date and time T2, and the content is canceled at the date and time T3 (a case where it is input that the symptoms have disappeared). At the date and time T2, a message “Continue?” is displayed on the touch panel 15, and the progress information is input with the same content as that of selecting this area. An example of the input method in this case or when the symptoms disappear will be described later with reference to FIGS. 13 and 14.

As described above, the input device 1 can be configured to store the symptoms and the severity of the symptoms that have been input in the past as initial information, inquire about the progress of the symptoms thereafter, and store the fact that the symptoms have changed from the input to the touch panel 15 as progress information. For example, with such a configuration, an input as exemplified in FIG. 3 can be performed.

In this case, the effective period of the tag information generated by referring to the progress information from the initial information can be T 1 to T 3, and the tag information is stored in association with the biological information acquired during the period. In this manner, the progress of the symptoms can be inquired, and the progress of the symptoms can be input by inputting the progress of the symptoms in response to the inquiry.

The timing of the transmission of the tag information in step S4 is not limited. When the initial information is transmitted before the progress information is input, the storage unit 22 can store the tag information in a state in which the tag information is associated with the biological information after the date and time indicated by the initial information. Further, as described above, for example, as 1 hour, the effective period of the health information is defined by a predetermined period, and the storage unit 22 may store the tag information in a state associated with the biological information for 1 hour from the date and time indicated by the initial information.

In any case, when the progress information corresponding to the initial information is transmitted, the information may be updated so that the tag information is stored in association with the biological information after the date and time indicated by the initial information and before the date and time indicated by the progress information.

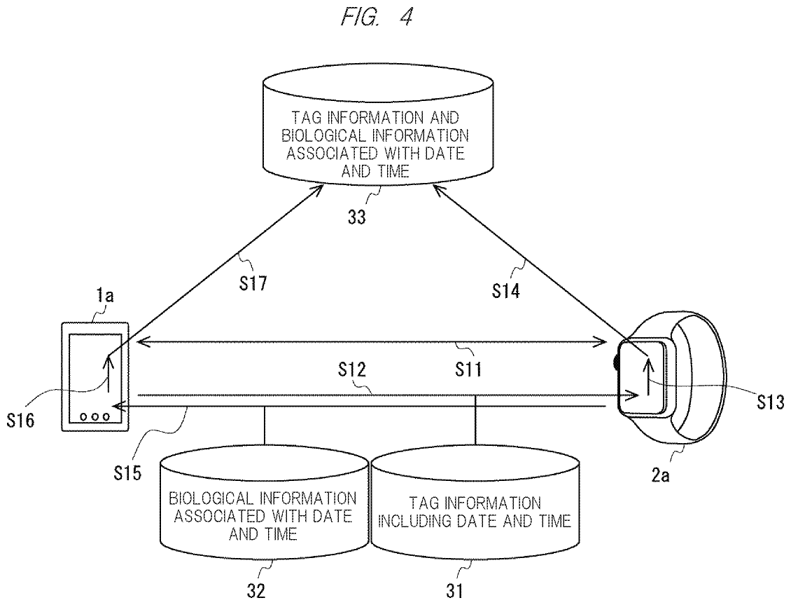

With reference to FIG. 4, an example of a flow of processing from input of health information to association between tag information and biological information in the input device 1 will be schematically described. FIG. 4 is a schematic diagram illustrating the flow of processing in the present system. In FIG. 4, a terminal device 1a is used as an example of the input device 1, and a wearable device 2a is used as an example of the collection device 2.

In this system, as shown in FIG. 4, the terminal device 1a and the wearable device 2a can synchronize the date and time with each other in step S11. This point is as described in step S2 of FIG. 2 and the like. In the present system, the terminal device 1a and the wearable device 2a can be configured to transmit information to each other (steps S12 and S15), and both can be configured to enable merging processing of tag information and biological information (steps S13 and S16).

Therefore, after the synchronization processing in step S11, the terminal device 1a transmits the tag information 31 including the date and time to the wearable device 2a (step S12), and the wearable device 2a can merge the tag information 31 and the internal biological information based on the date and time (step S13). The wearable device 2a may be configured to transmit the tag information and the biological information 33 associated with the date and time, which are the information of the merging result, to another device (for example, a server device on a PC or a cloud) (step S14). The PC may be any of a tablet type, a desktop type, and a notebook type, and may be a mobile phone similar to the terminal device 1a, including a mobile phone called a smart phone. The wearable device 2a can not only transmit the tag information and the biological information 33 to other devices but also store them in the internal storage unit 22.

After the synchronization processing in step S11, the wearable device 2a transmits the biological information 32 associated with the date and time to the terminal device 1a (step S15), and the terminal device 1a can merge the biological information 32 and the internal tag information based on the date and time (step S16). The terminal device 1a may be configured to transmit the tag information and the biological information 33 associated with the date and time, which are the information of the merge result, to another device (for example, a server device on the cloud) (step S17). The terminal device 1a not only transmits the tag information and the biological information 33 to the cloud, but also stores the tag information in the internal storage section.

As can be seen from the example of FIG. 4, the input device 1 may include the following receiving unit and storage unit, together with the input unit 11 and the generating unit 10a. This receiving unit is exemplified by the communication unit 14, and receives the biological information from the collection device 2 that acquires the biological information and collects the biological information in association with the second time information indicating the acquired date and time. The storage unit stores the tag information in a state in which the tag information is associated with the biological information based on the second time information and the first time information indicated by the tag information.

Also, as can be seen from the example of FIG. 4, the collection device 2 may have a storage unit 22 and a next receiving unit. The receiving unit is exemplified by the communication unit 23, and receives tag information including health information indicating a certain date and time and first time information indicating the certain date and time from the external input device 1.

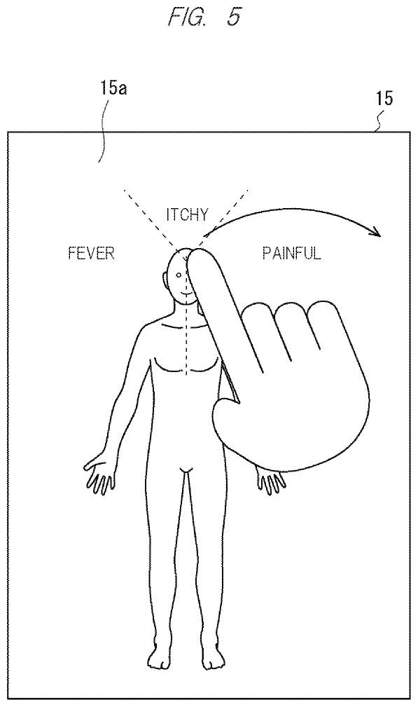

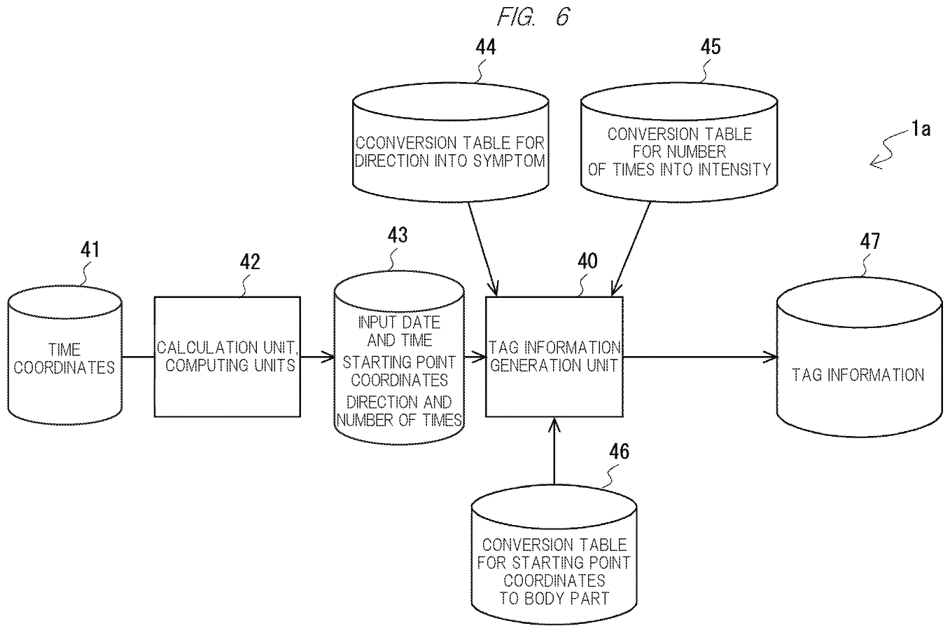

Next, an input example and a tag information generation example in the terminal device 1a will be described with reference to FIGS. 5 and 6. FIG. 5 is a conceptual diagram illustrating an example of a state in which an input is performed on the touch panel 15 of the terminal device 1a, and FIG. 6 is a block diagram illustrating a configuration example of a portion related to generation of tag information in the terminal device 1a.

As illustrated in FIG. 5, the terminal device 1a can display a GUI image 15a including a schematic diagram of a body and a symptom's name on the touch panel 15. As the symptom's name, it is preferable to display the symptom's name corresponding to the selected body part area when a part of the schematic diagram is selected. Though not shown, the schematic diagram of the body can also show the boundary line between the parts.

As shown in FIG. 6, the terminal device 1a can be provided with a tag information generating unit 40 that can read date and time information (time coordinates) 41, corresponds to a main part of the generating unit 10a, and generates tag information. In addition, the terminal device 1a may include a calculation unit 42 for calculating the input direction and the number of times, a conversion table 44 for converting the direction into the symptom, a conversion table 45 for converting the number of times into the intensity, and a conversion table 46 for converting starting point coordinates into the body part.

The tag information generation unit 40 inputs the input date and time output from the calculation unit 42, the starting point coordinates that became the starting point of contact (touch) on the touch panel 15 (touch was first detected), the direction in which the sliding operation was performed after the touch, and the number of times the sliding operation was performed within a predetermined time. Note that the input date and time may be only the input time.

The tag information generating unit 40 refers to the conversion table 44 based on the direction, and acquires information indicating a symptom indicated by the direction. The tag information generation unit 40 refers to the conversion table 45 based on the number of times to acquire information indicating the intensity (severity, degree) of the symptom indicated by the number of times, and refers to the conversion table 46 based on the starting point coordinates to acquire information indicating the biological part (body part). The tag information generation unit 40 generates the tag information 47 based on the various pieces of information acquired in this manner, including the input date and time. Instead of the conversion table 45, the terminal device 1a can have a conversion table for converting the length into the intensity, and calculate the length from the starting point coordinate to the end point coordinate, and acquire the intensity of the symptom by referring to the conversion table from the length.

With such a configuration, the terminal device 1a determines the area from the starting point coordinate at which the touch is first made on the touch panel 15, the symptom from the end point coordinate, the symptom confirmation date and time from the date and time of contact, and the severity (degree) of the symptom from the length of the starting point coordinate and the end point coordinate, respectively. The terminal device 1a stores the information acquired by the determination as the tag information 47 in an internal storage unit (not shown). By making such a determination, it is possible to input symptoms uniquely in real time with few operations.

The terminal device 1a shown in FIG. 6 can have, for example, a program for causing a control unit such as a CPU thereof to function as the calculation unit 42 and the tag information generation unit 40, and the program can also include conversion tables 44 to 46. The information 41 and 43 can be information read out to the buffer memory in the control unit, and the tag information 47 can be information stored in the storage unit in the terminal device 1a.

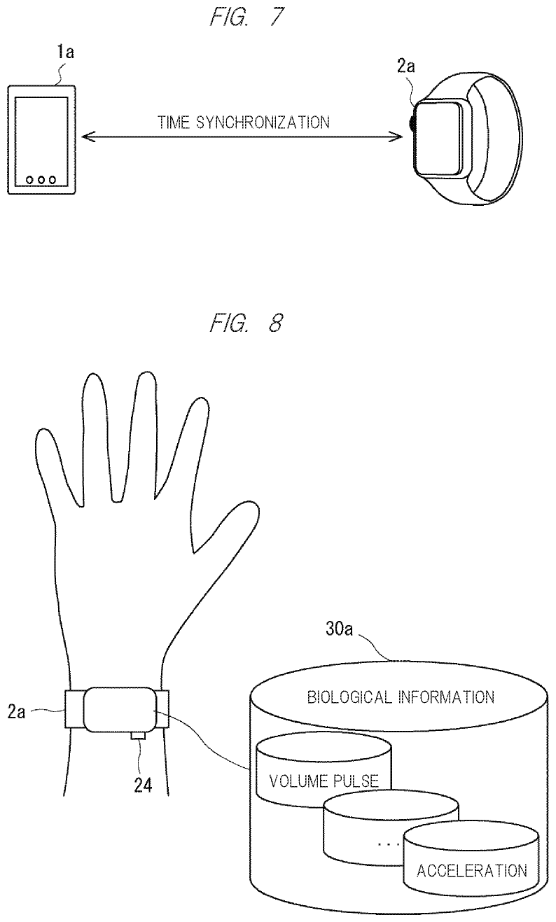

Next, a more specific processing example of the present system will be described with reference to FIGS. 7 to 21.

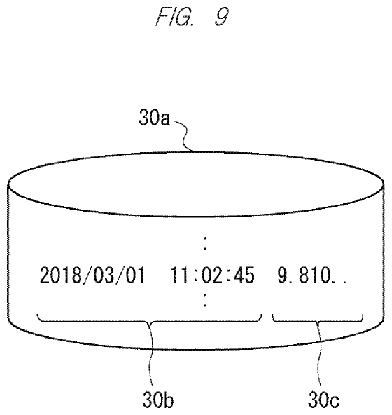

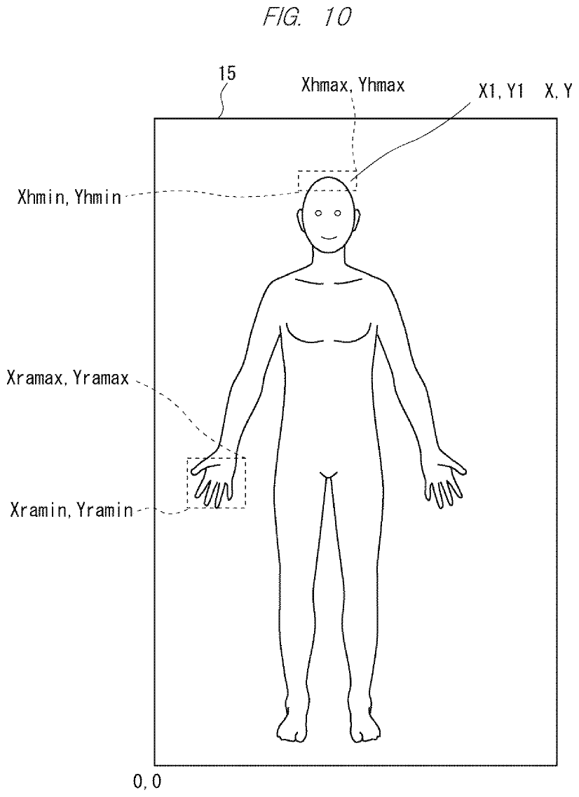

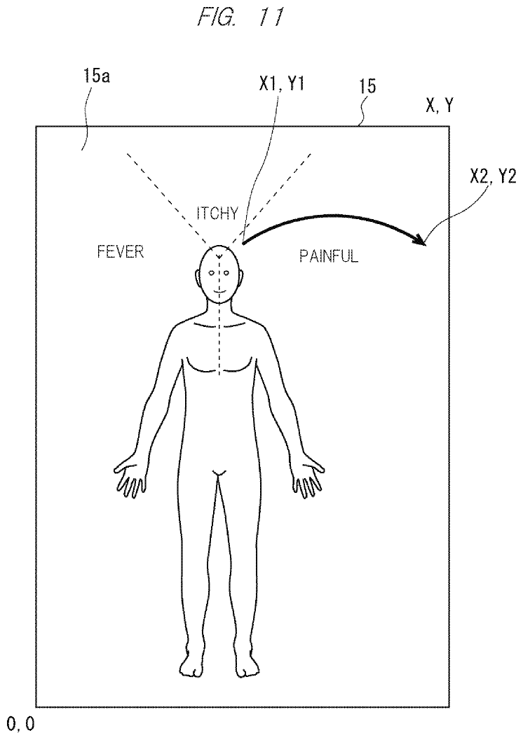

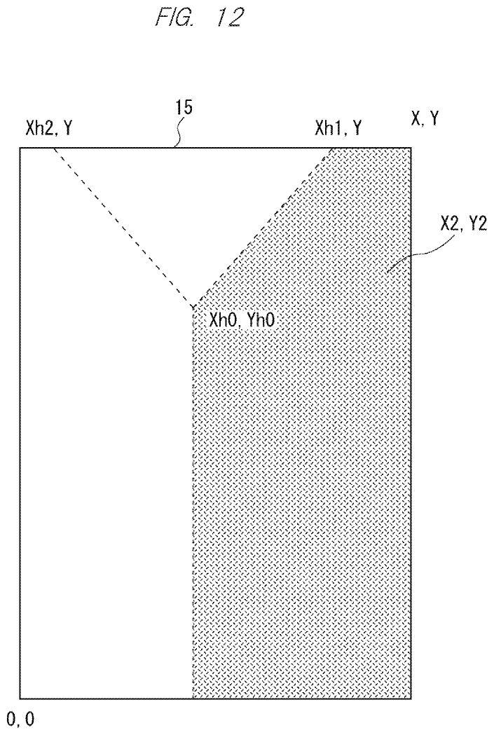

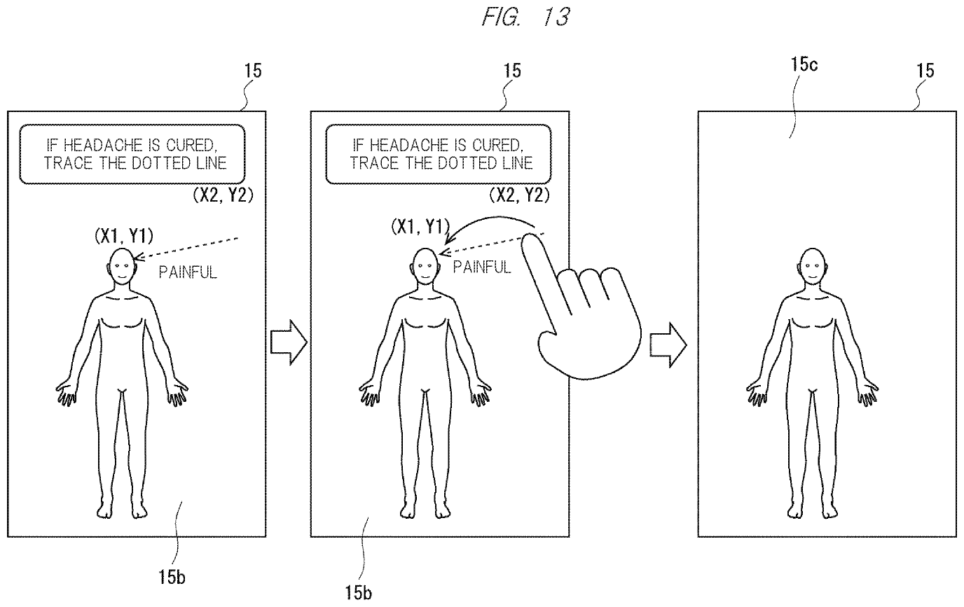

First, an example of input of health information will be described with reference to FIGS. 7 to 12. FIG. 7 is a conceptual diagram illustrating a state of time synchronization between the terminal device 1a and the wearable device 2a, FIG. 8 is a diagram illustrating an example of a wearable device 2a and the biological information collected therefrom, and FIG. 9 is a diagram illustrating an image of the data stored in the wearable device 2a. FIG. 10 is a diagram illustrating an example of the correspondence between the touch portion and the living body part, FIG. 11 is a diagram illustrating the distance between the starting point and the end point, and FIG. 12 is a diagram illustrating an example of the correspondence between the input end point coordinates and the symptoms.

After the terminal device 1a and the wearable device 2a are powered on and initialized, date and time synchronization (time synchronization) is performed between the wearable device 2a and the terminal device 1a using wireless communication technology or wired communication technology, as shown in FIG. 7. By performing such time synchronization, biological information and health information can be merged based on the time synchronized between the two devices. In FIG. 7, the state of time synchronization between the terminal device 1a and the wearable device 2a is shown, but the time may be synchronized between the tag information generating unit 40 in the terminal device 1a and the portion where the biological information is collected in the wearable device 2a.

Thereafter, as shown in FIG. 8, by attaching the wearable device 2a to the arm and pressing the measurement start button provided in the wearable device 2a, collection of the biological information 30a is started based on the sampling rate determined by the initial setting. The biological data 30a can be collected by, for example, measuring accelerations using a sensor such as MEMS (Micro Electro Mechanical Systems) or measuring volume pulse waves using an optical sensor. As shown in FIG. 9, the collected biological information 30a holds date and time information 30b and sensor data 30c as one set.

After the measurement is started in this manner, when the user feels headache, health information (in this case, initial information) is input using a program (application) for inputting health information which has been activated in advance in the terminal device 1a or has been activated at that time. Specifically, the user taps the head portion in the schematic diagram in the GUI image 15a displayed on the touch panel 15 as shown in FIG. 5 with a finger, and slides the finger in the direction displayed as painful.

The user can input the severity of the symptom by making the slide amount larger when the severity of the symptom is larger, making the slide amount smaller when the severity of the symptom is smaller, and sliding the finger. As shown in FIG. 10, the terminal device 1a refers to the conversion table 46 for converting the biological part from the starting point coordinates of FIG. 6 from the coordinates (X1, Y1) on the touch panel 15 that was pressed first, and converts the object to be the head. The conversion table 46 can be a conversion table in which, for example, the respective parts are converted into “head” in the case of Xhmin<X1<Xhmax and Yhmin<Y1<Yhmax in coordinates on the touch panel 15 shown in FIG. 10, and converted into “right hand fingertip” in the case of Xramin<X1<Xramax and Yramin<Y1<Yramax. As in this translation table, when the coordinates are processed, the coordinates (X1, Y1) are assumed to have a function to recognize the coordinates within a certain range as shown, for example, in FIG. 10.

Further, as shown in FIG. 11, assuming that the coordinates at which the user released the finger are (X2, Y2), the terminal device 1a refers to the conversion table 44 for converting the coordinates at which the finger was released (X2, Y2) into the symptoms from the direction of FIG. 5, and converts the symptoms into “painful”. The conversion table 44 can be a conversion table that converts the coordinates on the touch panel 15 shown in FIG. 12 to “painful” when Xh0<X2 and Y2≤(Y−Yh0)/(Xh1−Xh0)×Xh1+(Yh0×Xh1−Xh0×Y)/(Xh1−Xh0). As in the conversion table, when the coordinates are processed, the coordinates (X2, Y2) have a function of recognizing a certain range as shown in FIG. 12. Further, the range can be selected by the user by dividing and assigning the screen according to the necessary number of symptoms to be applied. It should be noted that it is desirable that which symptom is assigned where, the division ratio, and the like can be set by a user operation or the like.

Further, the terminal device 1a acquires the length, that is, the square root of ((X2−X1)²+(Y2−Y1)²)) from the starting point and the end point shown in FIG. 11, and converts the length into the severity of symptoms.

By a series of such operations, the terminal device 1a can hold the tag information as data in which the date and time of the first tap and the symptom and the severity of the input body part are set as one set. Here, as an example, the severity of the symptom is converted by the length between the starting point and the end point, but the length may be acquired by the trajectory from the suppression of the finger to the separation. Further, the severity of the symptoms is not limited to the length conversion, and as described with reference to FIG. 6, the number of times input for a predetermined period of time may be converted into the severity of the symptoms, or the pressure (pressing) by the finger may be converted into the severity of the symptoms. In this manner, by making the user input the health information by using the starting point, the end point, the trajectory, the press, and the like of the touch operation, it is possible to input the health information for generating the tag information uniquely and in real time with a small number of operations.

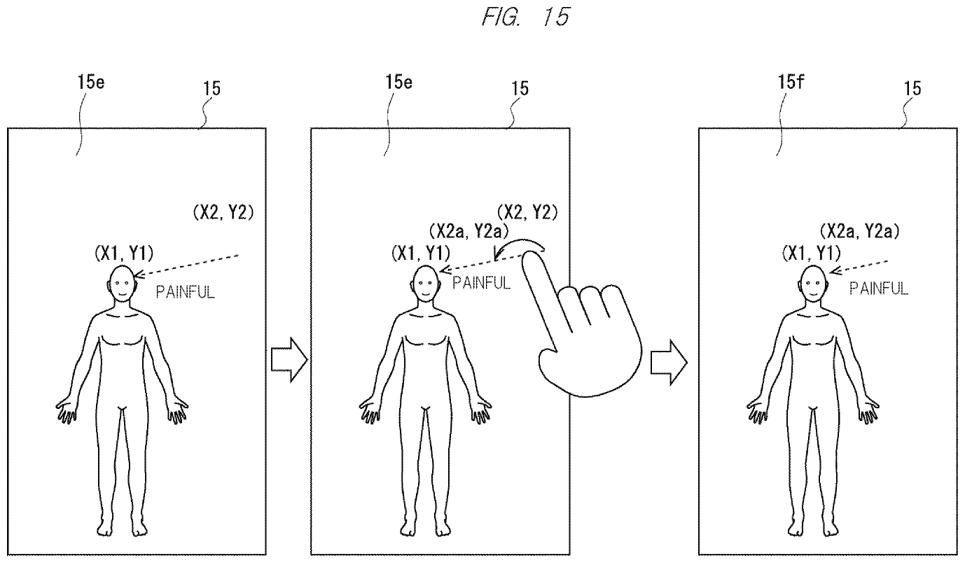

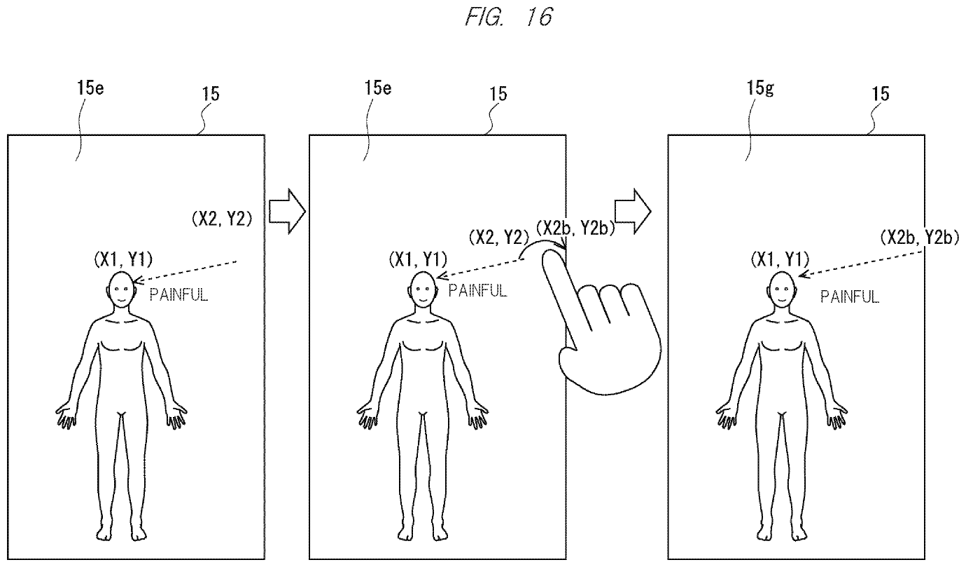

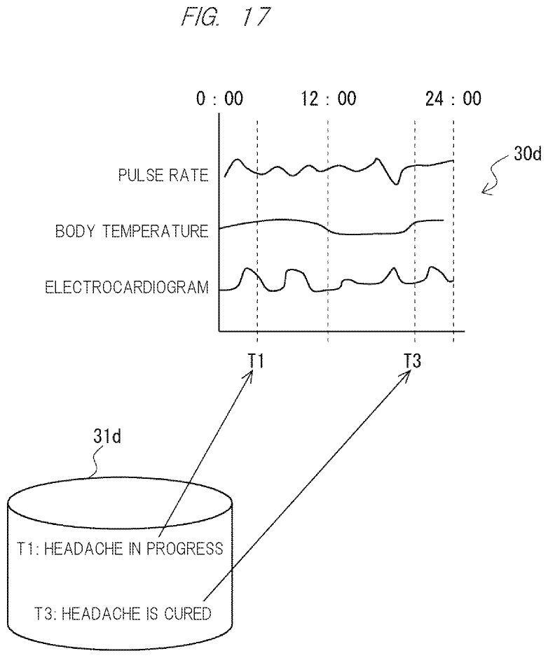

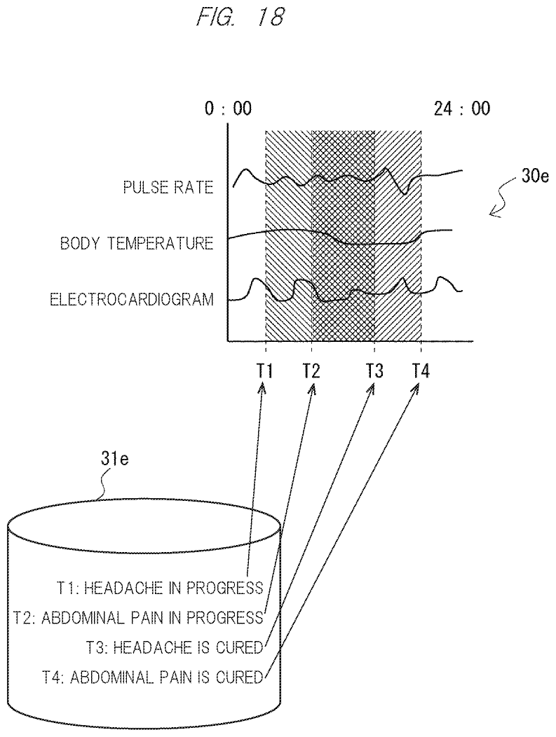

Next, an example of input of the progress information will be described with reference to FIGS. 13 to 19. FIG. 13 is a diagram illustrating an example of a series of GUI images including a GUI image for prompting input of symptom improvement (symptom end). FIG. 14 is a diagram illustrating an example of a series of GUI images for inputting symptom improvement. FIG. 15 is a diagram illustrating an example of a series of GUI images for inputting the degree at the time of symptom improvement, and FIG. 16 is a diagram illustrating an example of a series of GUI images for inputting the severity at the time of symptom deterioration. FIG. 17 is a conceptual diagram illustrating an example of biological information associated with health information, and FIG. 18 is a conceptual diagram illustrating another example of biological information associated with health information. FIG. is a diagram illustrating an example in which biological information is classified and stored for each piece of health information.

After inputting the health information (initial information) as described with reference to FIGS. 10 to 12, the terminal device 1a displays the GUI image 15b including the inquiry message on the touch panel 15 as shown in FIG. 13. The GUI image 15b is preferably displayed as a pop-up image after a predetermined period of time has elapsed. This example is an example after health information indicating headache is input, and “If headache is cured, trace the dotted line” is displayed as a message. According to this inquiry, the user can input the completion of the symptom to the terminal device 1a by performing an operation of tracing from the end point of the symptom input toward the starting point. When such a completion input is made, the terminal device 1a displays a GUI image 15c for inputting initial information on the touch panel 15. In the GUI image 15b, similarly to the example of FIG. 3, a message of “Continue?” can be included, and when the area of the message is selected, the health information (progress information) having the same content as the preceding health information is input.

In the GUI image 15b of FIG. 13, an example including an inquiry message has been described, but the inquiry method is not limited to this. For example, information input in the past such as the GUI image 15d shown in FIG. 14, that is, information indicating a body part, a symptom, and a severity, may be displayed by a dotted line, and the symptom may be displayed by a schematic diagram. Alternatively, input may be prompted by another method such as simply displaying the input information by a dotted line as in the GUI image 15e shown in FIG. 15.

FIGS. 13 and 14 show input examples when the symptoms are completed. However, as shown by the slide operation on the GUI image 15e in FIG. 15, the previous input information may be displayed by a dotted line, and the improvement of the severity of the symptom may be input by tracing the dotted line in the direction of the area. The terminal device 1a displays the GUI image 15f in which the dotted line is shortened in accordance with the progress information input by the slide operation.

Alternatively, as shown in the slide operation with respect to the GUI image 15e of FIG. 16, the deterioration of the symptom can be input by tracing in the extending direction. The terminal device 1a displays the GUI image 15f in which the dotted line is lengthened in accordance with the progress information input by the slide operation. In the example of FIGS. 15 and 16, it is assumed that the tracing starts from the end point input in the past, but it is also possible to configure so that the input of the symptom progress information can be started from the dotted line portion in the middle.

After the progress information is input in this manner, a tag information transfer button (not shown) similar to the measurement start button 24 of the wearable device 2a shown in FIG. 8 is pressed. It is also possible to judge that the tag information transfer button has been pressed by pressing the measurement start button 24 for a long time, for example. When the tag information transfer button is pressed, the wearable device 2a sends a transmission request to the terminal device 1a using the wireless communication technology, and the terminal device 1a transmits the tag information 31 to the wearable device 2 a as shown in FIG. 4. As illustrated in FIG. 17, the wearable device 2a creates data 30d in which tag information 31d associated with the date and time is merged with the biological information associated with the date and time of the wearable device, based on the date and time, and stores it in an internal non-volatile memory (corresponding to the storage unit 22 in the collection device 2). The tag information 31d shown in FIG. 17 indicates that headache continued for the periods T1 to T3. In this case, the data 30d shown in FIG. 17 is data in which tag information indicating headache (effective periods T1 to T3) is associated with the pulse rate, body temperature, and electrocardiogram of the effective periods T1 to T3.

In addition, as shown in FIG. 18, the terminal device 1a may input health information about a plurality of symptoms in chronological order to generate tag information 31e for each combination of symptoms and body parts. In this case, as shown in FIG. 18, the wearable device 2a merges the tag information 31e of each combination of symptoms and body parts into the biological information to create the data 30e and stores the data in the internal non-volatile memory. The tag information 31e shown in FIG. 18 indicates that the headache (headache) continued for the periods T1 to T3, and the abdominal pain continued for the periods T2 to T4. In this case, the data 30e is data in which tag information indicating headache (effective periods T 1 to T 3) and tag information indicating abdominal pain (effective periods T2 to T4) are associated with the pulse, body temperature, and electrocardiogram in the respective effective periods.

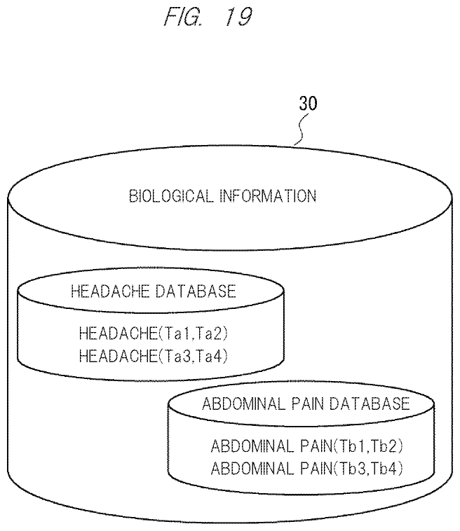

Thereafter, the wearable device 2a is connected to another device such as a PC or a server device using a wireless communication technique or a wired communication technique, and can store the health information and the biological information in the other device as merged information. Further, as shown in FIG. 19, the database 30 with which the tag information is associated can be stored on the other device in a state in which the biological information is classified for each health information (for each combination of symptoms and body parts).

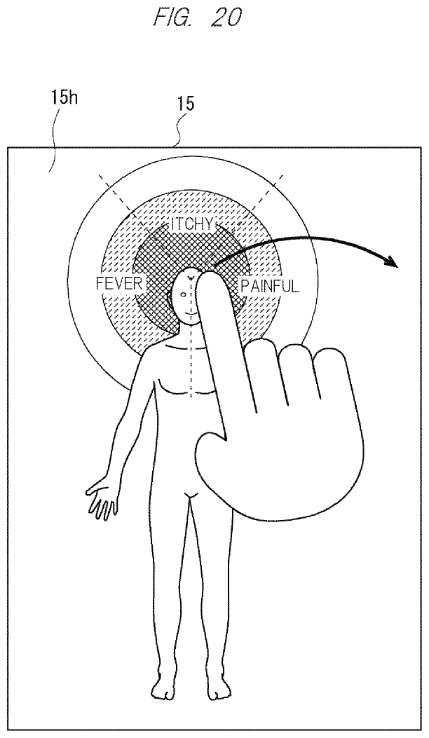

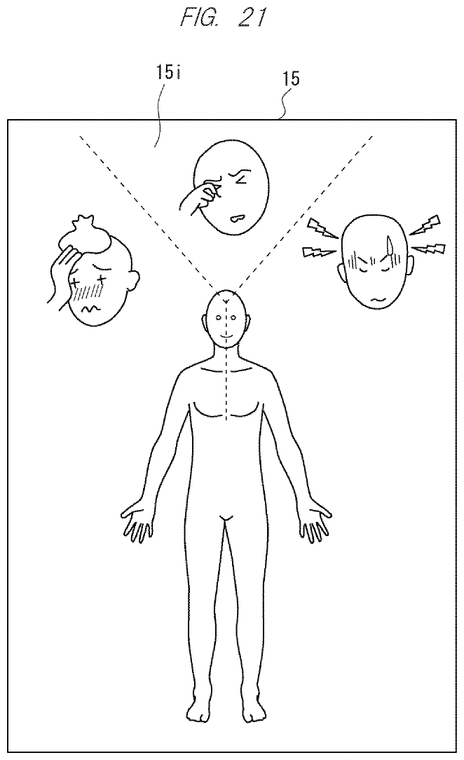

Next, another example of the GUI image will be described with reference to FIGS. 20 and 21. FIGS. 20 and 21 are diagrams illustrating another example of the GUI image shown in FIG. 5. As shown in FIG. 20, the terminal device 1a can also display a GUI image 15h in which color coding is performed to indicate the severity of symptoms on the touch panel 15. As a result, it is possible to define the stage of the severity of symptoms, and the user can input the corresponding severity in accordance with the defined stage, so that it is possible to easily input the severity suitable for the user's intention.

In the above example, Japanese such as “painful” is basically displayed as the symptom's name, but as shown in FIG. 21, the terminal device 1a can display a GUI image 15i representing the symptom's name in a schematic diagram independent of the language on the touch panel 15 in order to input health information.

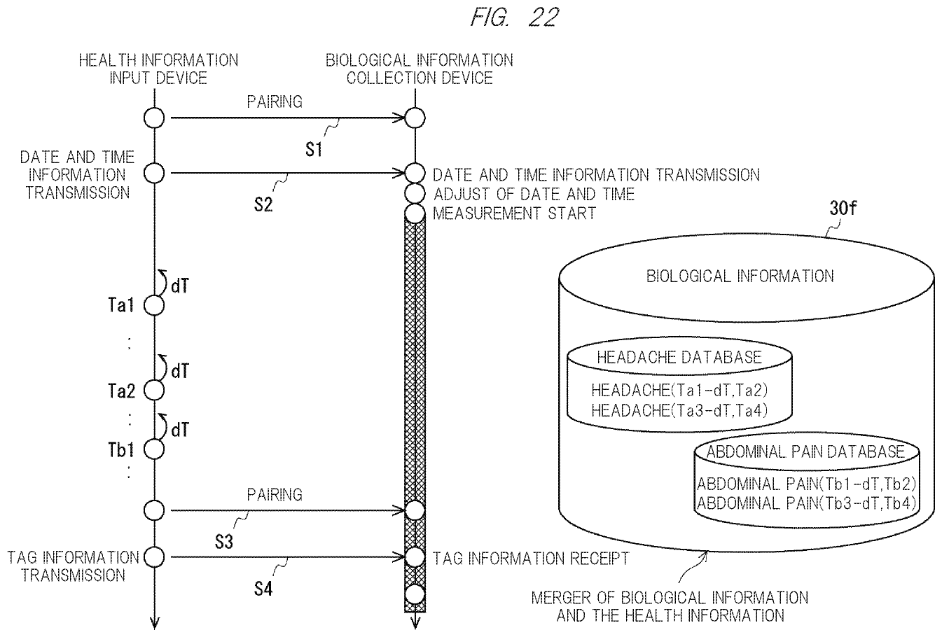

A biological information collection system according to Embodiment 2 will be described with reference to FIG. 22. FIG. 22 is a flowchart illustrating an example of processing in the biological information collection system according to the present embodiment. The present embodiment will be mainly described focusing on differences from the first embodiment.

The storage unit 22 of the collection device 2 according to the present embodiment retraces the first time information indicated by the tag information for a predetermined period of time, and stores the tag information in a state in which the tag information is associated with the biological information. That is, the collection device 2 according to the present embodiment associates the first time information indicated by the tag information with the biological information in a state in which the first time information is traced back for a predetermined time.

As shown in FIG. 22, in the biological information system according to the present embodiment, steps S1 to S4 can be executed similarly to the processing of FIG. 2. However, in the processing of FIG. 2, the time at which the health information is input is used as a starting point, whereas in the present embodiment, the following duration of the health condition (the effective period of the tag information) is adopted in order to more accurately specify the health condition. That is, the effective period of the tag information in the present embodiment is set to a period beginning from the date and time retreated from the time at which the health information is input by the predetermined time dT which is a duration from the occurrence of the symptom to the input, until the stop of the symptom (or the date and time retreated from the time at which the health information as the stop of the symptom is input by the predetermined time dT).

Therefore, in the database 30f of the biological information with which the tag information is associated, for example, tag information of the effective periods Ta1-dT to Ta2 and tag information of the effective periods Ta3-dT to Ta4 exist for the tag information with which the health information indicates headache. The respective pieces of tag information are associated with the biological information of the respective effective periods. Tag information Ta1 of headache whose effective period is determined by the initial time information (Ta1) and the progress time information (Ta2) will be described as an example. The tag information Ta1 of the headache is stored in a state in which the initial time information (Ta1) indicated by the tag information Ta1 is retraced for a predetermined time (dT), and in a state in which it is associated with the biological information of the corresponding time zone, that is, the biological information of the period (Ta1-dT, Ta2) indicated by the second time information. Similarly, regarding abdominal pain, tag information of the effective periods Tb1-dT to Tb2 and tag information of the effective periods Tb3-dT to Tb4 exist, and the respective tag information is associated with the biological information of the respective effective periods. Also, in the example of FIG. 22, similarly to the example of FIG. 2, the biological information can be stored as individual databases collectively for each piece of health information.

In the present embodiment, it is possible to more accurately specify the health condition. In addition, it is preferable that the collection device 2 is configured so that the predetermined period dT can be changed by a user operation or the like. The collection device 2 can also vary the constant period dT depending on the combination of symptoms and body parts. For example, it is assumed that a more intense headache than a weaker headache cannot be tolerated in time. However, such a configuration makes it possible to adjust the start time of the symptom to be constant to some extent regardless of the symptom or body part by, for example, setting the constant period dT for the input for the strong headache to be shorter than the constant period dT for the input for the weak headache.

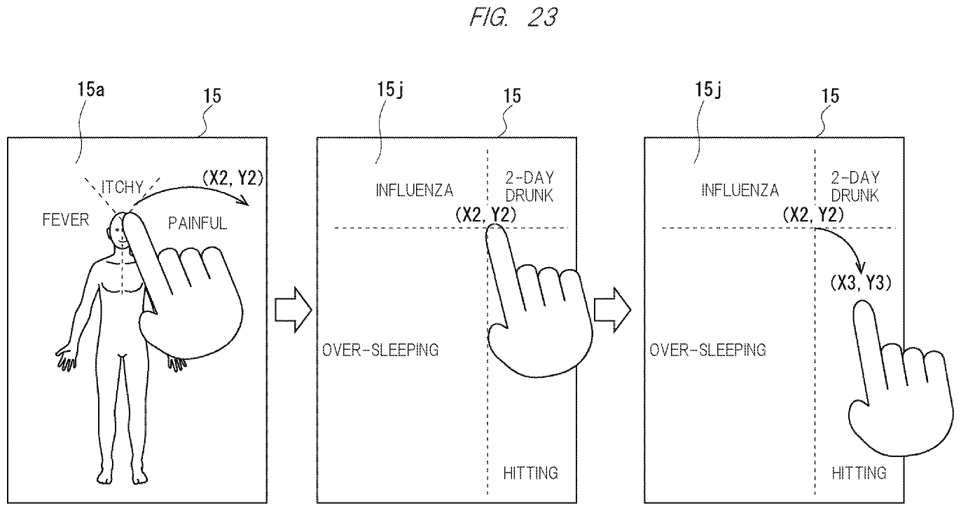

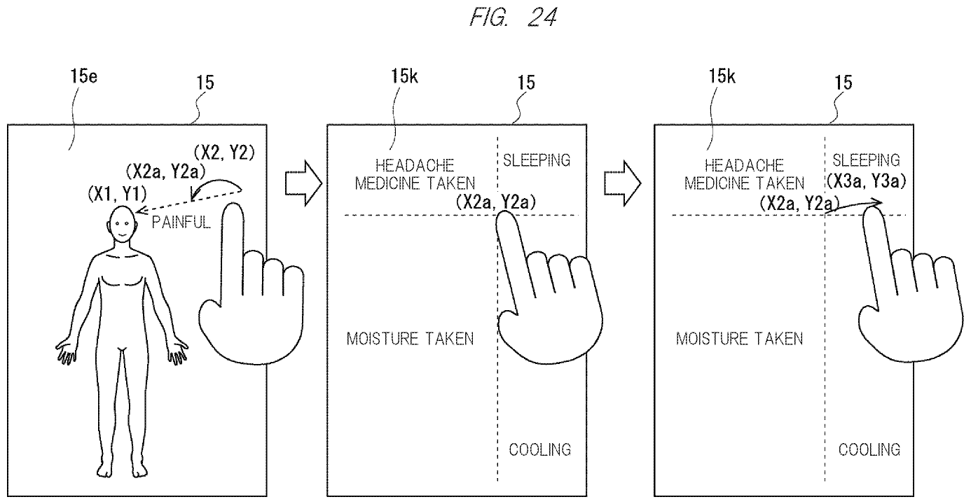

A biological information collection system according to a third embodiment will be described with reference to FIGS. 23 and 24. FIG. 23 is a diagram illustrating an example of a series of GUI images for inputting a cause of a symptom, and FIG. 24 is a diagram illustrating an example of a series of GUI images for inputting a reason for improvement of a symptom. The present embodiment will be described mainly with respect to differences from Embodiments 1 and 2.

The display control unit 13 in the input device 1 according to the present embodiment causes the display unit 12 to display a GUI image for inputting a cause that the user presumes to be the cause of the symptom or the reason of the recovery of the symptom. For example, the GUI image can be displayed by interrupting the sliding operation at a position indicating the severity of symptoms from the GUI image 15a in which the body part area is selected, as shown in the GUI image 15j illustrated in FIG. 23.

In this example, when a symptom is input in the GUI image 15a and stopped at coordinates (X2, Y2) indicating the severity of the symptom for one second, for example, as shown in FIG. 23, the input unit 11 regards the coordinates as the end point coordinates and inputs the severity information.

At the time of the input, the display control unit 13 displays a list of causes of symptoms (or actions causing the causes) in a selectable state in the next GUI image 15j prepared in advance. The user can enter the cause of the input unit 11 by tracing the touch panel 15 in the direction of the displayed cause.

In the GUI image 15j, as four causes of headache, “influenza”, “2-day drunk”, “over-sleeping”, or “hitting” is displayed in a selectable manner. When the user performs a sliding operation up to the coordinates (X 3, Y 3), for example, as shown in FIG. 23, the input unit 11 of the input device 1 can input the cause (in this case, “hitting”) corresponding to the coordinates as part of the health information. Since the relative coordinates of the coordinates (X2, Y2) and the coordinates (X3, Y3) are X2<X3 and Y2>Y3, the input unit 11 can enter “hitting” as the cause. In this manner, the input unit 11 inputs the information indicating the cause input in the GUI image 15j as at least a part of the health information.

In addition, as shown in FIG. 24, the same input can be made for the symptom improvement (recovery of the symptom), and the reason of the symptom improvement can be input. More specifically, at the stage of inputting the recovery of the symptom in the GUI image 15e, when stopping for one second, for example, at the coordinates (X2a, Y2a) indicating the degree of recovery of the symptom as shown in FIG. 24, the input unit 11 regards the coordinates as the end point coordinates and inputs the information on the degree of recovery.

At the time of the input, the display control unit 13 displays a list of the reasons of the recovery of the symptoms (or actions causing the reasons) in a selectable state in the next GUI image 15k prepared in advance. The user can enter the reason to the input unit 11 by tracing the touch panel 15 in the direction of the displayed cause.

In the GUI image 15k, one of the four reasons of recovery of the headache is displayed so as to be selectable, i.e., “headache medicine taken,” “sleeping,” “moisture taken,” and “cooling.” When the user performs a slide operation up to the coordinates (X3a, Y3a) as shown in FIG. 24, for example, the input unit 11 of the input device 1 can input the reason (in this case, “sleeping”) corresponding to the coordinates as part of the health information. Since the relative coordinates of the coordinates (X2a, Y2a) and the coordinates (X3a, Y3a) are X2a<X3a and Y2a>Y3a, the input unit 11 can enter the reason as “sleeping.” In this manner, the input unit 11 inputs the information indicating the reason input in the GUI image 15k as at least a part of the health information.

In the present embodiment, the cause of the symptom or the reason of the recovery of the symptom can be displayed in a selectable manner by temporarily stopping the input of the health information or the like, and the user can input the cause. Therefore, according to the present embodiment, it is possible to input not only the date and time, the body part, the symptom, and the severity, but also the cause or reason thereof, and it is possible to input the symptom and the action (cause) uniquely in real time with a small number of operations.

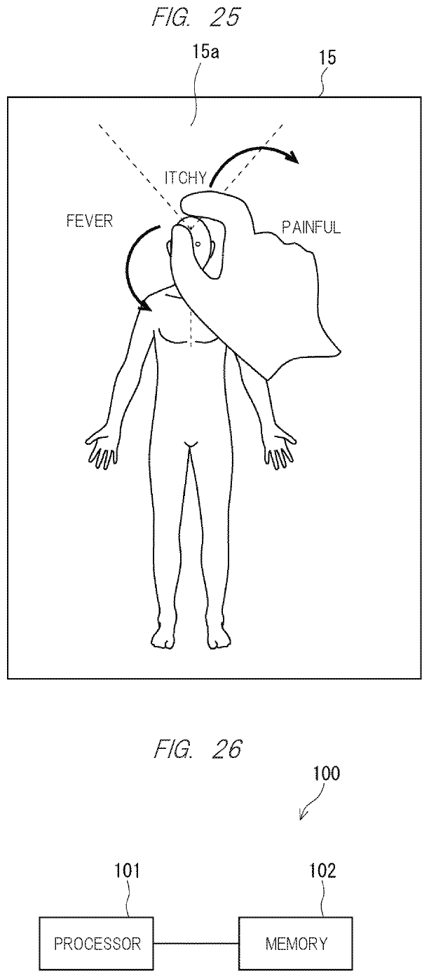

The biological information collection system according to the fourth embodiment is described with reference to FIG. 25. FIG. 25 is a conceptual diagram illustrating another example of a state in which input is performed on the touch panel 15, and shows an example of a state in which two pieces of health information are simultaneously input. The present embodiment will be described mainly with respect to differences from Embodiments 1 to 3.

The display control unit 13 in the input device 1 according to the present embodiment causes the display unit 12 to display a GUI image capable of accepting simultaneous input of a plurality of symptoms. In this case, the input unit 11 inputs each piece of information indicating the plurality of symptoms as at least a part of individual health information. Such simultaneous input can be easily accepted particularly by providing the touch panel 15.

Such a GUI image may be the GUI image 15a shown in FIG. 25, similarly to the GUI image 15a shown in FIG. 5, but the input unit 11 (touch sensor in the touch panel 15) may be configured to receive simultaneous input. The input unit 11 in this example inputs two symptoms of “fever” and “painful” at the same time by a pinch-out operation by the user as shown in FIG. 25. The operation method is not limited to the pinch-out operation, and three or more symptoms can be input simultaneously. According to the present embodiment, since a plurality of symptoms can be input simultaneously, the operation time can be shortened.

Each device in the biological information collection system according to Embodiments 1 to 4 can have the following hardware configuration. FIG. 26 is a diagram illustrating an example of a hardware configuration included in the device in the biological information collection system.

The device 100 shown in FIG. 26 includes a communication interface (not shown) and, if necessary, a sensor, and includes a processor 101 and a memory 102. The various devices described in the above embodiments are realized by the processor 101 reading and executing a control program stored in the memory 102. That is, the control program is a program for causing the processor 101 to function as a target device.

Some or all of the above embodiments may also be described as the following appendix, but the present invention is not limited to the following.

(Appendix 1) A biological information collection system, comprising: an input device for inputting health information indicating a health condition of a certain date and time and first time information indicating the certain date and time; and a biological information collection device for acquiring and collecting biological information in association with second time information indicating an acquired date and time; a generation unit for generating tag information based on the health information and the first time information; and a storage unit for storing the tag information in association with the biological information based on the second time information and the first time information indicated by the tag information.

(Appendix 2) The biological information collection system according to Appendix 1, wherein the input device has a notification unit that performs notification to prompt input of progress information indicating a progress of health condition as the health information based on the already input health information.

(Appendix 3) The biological information collection system according to Appendix 1, wherein the input device is configured to input initial information as a type of health information indicating a first health condition for a symptom or a body part, and initial time information as the first time information for the initial information, and a progress information as a type of health information indicating a progress from the first health condition, and a time progress information as the first time information for the progress information, wherein the generating unit is configured to generate the tag information for the initial information based on the initial information, the initial time information, and the progress time information.

(Appendix 4) The biological information collection system according to Appendix 1, wherein the input device includes a display unit, a display control unit that displays an area indicating a body part in a selectable state, and displays an area indicating a symptom that may occur in an area corresponding to the selected area in a selectable state when the area is selected, on the display unit, and inputs information indicating a body part and a symptom corresponding to the selected areas in the user interface image as the health information.

(Appendix 5) The biological information collection system according to Appendix 1, wherein the input device includes a display unit, a display control unit that displays a user interface image for displaying an area indicating symptoms in a selectable state, and displaying an area indicating a body part corresponding to the selected area in a selectable state when the area indicating symptoms is selected, on the display unit, and inputs information indicating symptoms and a body part corresponding to the selected areas in the user interface image as the health information.

(Appendix 6) The biological information collection system according to Appendix 1, wherein the input device includes a display unit, a display control unit that displays a user interface image that displays an area indicating a body part in a selectable state, and displays an area indicating a symptom that may occur in an area corresponding to the selected area in a selectable state, when the area is selected, and determines a severity of a symptom in accordance with a distance from a selected position or a predetermined position in the area indicating a body part to a selected position in the area indicating a symptom, on the display unit, and inputs information indicating a body part and a symptom corresponding to the selected areas in the user interface image, and information indicating the severity, as the health information.

(Appendix 7) The input device includes a display unit, a display control unit that displays an initial user interface image that displays an area indicating a body part in a selectable state, and when the area is selected, an area indicating a symptom that may occur in an area corresponding to the selected area in a selectable state on the display unit, and determines a severity of a symptom in accordance with a distance from a selected position or a predetermined position in the area indicating a body part to a position finally selected in the area indicating a symptom, and inputs information indicating a body part and a symptom corresponding to the selected area in the initial user interface image as a part of initial information indicating an initial health condition as a kind of health information, and displays a progress input user interface image that displays information indicating the severity in the initial information in a changeable state on the display unit, and inputs progress information indicating a changed severity in the progress input user interface image and the information indicating a body part and a symptom corresponding to the selected areas in the initial user interface image as the health information indicating a progress from the initial health condition.

(Appendix 8) The biological information collection system according to Appendix 1, wherein the input device includes a display unit and a display control unit that displays a user interface image for inputting a cause to be presumed by the user to be a cause of symptom or reason of symptom recovery, and inputs information indicating the cause or reason in the user interface image as the health information.

(Appendix 9) The biological information collection system according to Appendix 1, wherein the input device comprises a display unit and a display control unit for displaying a user interface image capable of receiving simultaneous inputs for a plurality of symptoms on the display unit, each of the information representing the plurality of symptoms being inputted as the health information respectively.

(Appendix 10) The biological information collection system according to Appendix 1, wherein the storage unit retraces the first time information indicated by the tag information for a predetermined time, and stores the tag information in a state in which the tag information is associated with the biological information.

(Appendix 11) The biological information collection system according to Appendix 1, wherein the health information includes information indicating symptoms and body parts, and the biological information is stored for each combination of symptoms and body parts according to the health information indicated by the tag information.

(Appendix 12) an input unit for inputting health information indicating a health condition of a certain date and time and first time information indicating the certain date and time; a generation unit for generating tag information based on the health information and the first time information; a reception unit for receiving the biological information from a biological information collection device for acquiring the biological information and collecting the biological information in association with the second time information indicating the acquired date and time; and a storage unit for storing the tag information in association with the biological information based on the second time information and the first time information indicated by the tag information.

(Appendix 13) A biological information collection device, comprising: a storage unit for acquiring biological information and storing the biological information in a state of being associated with first time information indicating an acquired date and time; and a receiving unit for receiving tag information including health information indicating a health condition of a certain date and time and second time information indicating the certain date and time from an external input device, wherein the storage unit stores the tag information in a state of being associated with the biological information based on the first time information and the second time information indicated by the tag information.

(Appendix 14) A biological information collection method, comprising: an input step of inputting health information indicating a health condition of a certain date and time and first time information indicating the certain date and time; and a biological information collection step of acquiring biological information and collecting the biological information in association with second time information indicating the acquired date and time; and a generation step of generating tag information based on the health information and the first time information, and a storage step of storing the tag information in association with the biological information based on the second time information and the first time information indicated by the tag information.

(Appendix 15) The method for collecting biological information according to Appendix 14, wherein said input step comprises a step of providing a notification on the basis of the previously input health information that prompts the input of a progress health information indicating a progress of the health condition as the health information.

(Appendix 16) The biological information collection method according to Appendix 14, wherein the input step comprises: inputting initial information as the health information indicating an initial health condition of a certain symptom or a certain body part, initial time information as the first time information about the initial information; inputting progress information as the health information indicating a progress from the initial health condition, and progress time information as the first time information about the progress information; and generating the tag information about the initial information based on the initial information, the initial time information, and the progress time information.

(Appendix 17) The method for collecting biological information according to Appendix 14, wherein said input step comprises a display control step for displaying on a display unit a user interface image displaying an area indicating a body part in a selectable state and, when the area is selected, an area indicating a symptom that may occur in the selected area in a selectable state; and an information input step of inputting information indicating a body part and a symptom corresponding to the selected areas in said user interface image as the health information.

(Appendix 18) The method for collecting biological information according to Appendix 14, wherein the input step includes a display control step for displaying in a display unit a user interface image in which an area indicating a symptom is displayed in a selectable state and, when the area is selected, an area indicating a body part corresponding to the selected area is displayed in a selectable state, and inputting information indicating a symptom and a body part corresponding to the areas selected respectively in the user interface image as the health information.

(Appendix 19) The biological information collecting method according to Appendix 14, wherein the input step includes a display control step of displaying a user interface image that displays an area indicating a body part in a selectable state, and, when the area is selected, displays an area indicating a symptom in a selectable state for an area corresponding to the selected area on a display unit, and determines a severity of a symptom according to a distance from a selected position or a predetermined position in the area indicating a body part to a selected position in the area indicating the symptom, and an information input step of inputting information indicating a body part and a symptom corresponding to the selected areas and information indicating the severity as the health information.

(Appendix 20) The input step includes a display control step of displaying a initial user interface image that displays an area indicating a body part in a selectable state and, when the area is selected, and displays an area indicating a symptom that may occur in an area corresponding to the selected area in a selectable state on a display unit, and determines a severity of a symptom in accordance with a distance from a selected position or a predetermined position in the area indicating a body part to a position finally selected in the area indicating a symptom, and an information input step of inputting information indicating a body part and a symptom corresponding to the selected areas as a part of health information indicating an initial health condition, and displaying a progress input user interface image that displays information indicating the severity in the initial information in a changeable state on the display unit, and a progress information input step of inputting progress information indicating a changed severity in the progress input user interface image and the information indicating a body part and symptoms corresponding to the selected area in the initial user interface image as a part of the health information indicating progress from the initial health condition.

(Appendix 21) The method for collecting biological information according to Appendix 14, wherein said input step includes a display control step for displaying a user interface image on a display unit for inputting a cause that a user infers is the cause of the symptom or the reason of the recovery of the symptom, and an information input step of inputting information indicating the cause or reason input in said user interface image as the health information.

(Appendix 22) The method for collecting biological information according to Appendix 14, wherein the input step includes a display control step for displaying a user interface image capable of receiving simultaneous inputs for a plurality of symptoms on a display unit, and an information input step of inputting each of the information indicating the plurality of symptoms as a portion of the health information respectively.

(Appended 23) The method for collecting biological information according to Appendix 14, wherein the storage step retraces for a predetermined period of time the first time information represented by the tag information and stores the tag information in a state associated with the biological information.

(Appendix 24) The method for collecting biological information according to Appendix 14, wherein said health information comprises information indicating symptoms and body parts, and wherein said storage step stores said biological information for each combination of symptoms and body parts in accordance with said health information indicated by said tag information.

(Appendix 25) An input method, comprising: an input step of inputting health information indicating a health condition of a certain date and time and first time information indicating the certain date and time; a generating step of generating tag information based on the health information and the first time information; a receiving step of receiving the biological information from a biological information collection device that acquires the biological information and collects the biological information in association with the second time information indicating the acquired date and time; and a storage step of storing the tag information in association with the biological information based on the second time information and the first time information indicated by the tag information.

(Appendix 26) A biological information collection method, comprising: a storage step of acquiring biological information, storing the biological information in a state of being associated with first time information indicating an acquired date and time; and a receiving step of receiving tag information including health information indicating a health condition of a certain date and time and second time information indicating the certain date and time from an external input device, wherein the storage step stores the tag information in a state of being associated with the biological information based on the first time information and the second time information indicated by the tag information.

(Appendix 27) A program for executing the steps of: inputting health information indicating a health condition of a certain date and time and first time information indicating the certain date and time into a computer; generating tag information based on the health information and the first time information; receiving the biological information from a biological information collection device that acquires the biological information and collects the biological information in association with the second time information indicating the acquired date and time; and storing the tag information in association with the biological information based on the second time information and the first time information indicated by the tag information.

(Appendix 28) The program according to Appendix 27, wherein the input step comprises a notification step providing a notification, based on the health information already input, that prompts an input of progress information indicating a progress of the health condition as a kind of health information.

(Appendix 29) The program according to Appendix 27, wherein the input step comprises: inputting an initial information as a kind of health information indicating a first health condition for a symptom or a body part; and an initial time information as the first time information for the initial information; inputting a progress information as a kind of health information indicating a progress from the first health condition; and inputting an progress time information as the first time information for the progress information; and generating the tag information for the initial information based on the initial information and the initial time information and the progress time information.

(Appendix 30) The program according to Appendix 27, wherein the input step includes a display control step of displaying on a display unit a user interface image that displays an area indicating a body part in a selectable state and, if the area is selected, displays an area indicating a symptom that may occur in an area corresponding to the selected area in a selectable state, and an information input step of inputting information indicating a body part and a symptom corresponding to the selected areas in the user interface image as the health information.

(Appendix 31) The program according to Appendix 27, wherein the input step comprises a display control step for displaying in a display unit a user interface image which displays an area indicating a symptom corresponding to a selected area in a selectable state and an area indicating a body part corresponding to a selected area in a selectable state when the area is selected, and an information input step of inputting information indicating a symptom and a body part corresponding to a selected area in the user interface image as a part of the health information.

(Appendix 32) The program according to Appendix 27, wherein the input step includes a display control step of displaying an area indicating a body part in a selectable state and displaying an area indicating a symptom in a selectable state for an area corresponding to a selected area when the area is selected, on a display unit, determining a severity of a symptom in accordance with a distance from a selected position in the area indicating a body part or a predetermined position in the area indicating a body part to a selected position in the area indicating a symptom, and an information input step of inputting information indicating an area and a symptom corresponding to the selected area in the user interface image and information indicating the severity as at least a part of the health information.

(Appendix 33) The input step includes a display control step of displaying an initial user interface image that displays an area indicating a body part in a selectable state and, when the area is selected, displays an area indicating a symptom that may occur in an area corresponding to the selected area in a selectable state on a display unit, and determines a severity of a symptom in accordance with a distance from a selected position or a predetermined position in the area indicating a body part to a position finally selected in the area indicating a symptom, and an information input step of inputting information indicating the body part and the symptom corresponding to the selected areas as initial information indicating an initial health condition as health information, and the display control step further displaying a progress input user interface image that displays information indicating the severity in a state in which information indicating the severity in the initial information in a changeable state on the display unit, and an progress information input step of inputting progress information indicating a changed severity in the progress input user interface image and the information indicating a body part and a symptom corresponding to the selected area in the initial user interface image as progress information as the health information indicating a progress from the initial health condition.

(Appendix 34) The program according to Appendix 27, wherein the input step includes a display control step for displaying a user interface image on a display unit for inputting a cause that the user infers to be the cause of the symptom or reason of recovery of the symptom, and a cause information input step of inputting information indicating the cause input in the user interface image as the health information.

(Appendix 35) The program according to Appendix 27, wherein the input step includes a display control step for displaying a user interface image capable of receiving simultaneous inputs for a plurality of symptoms on a display unit, and a simultaneous information input step of inputting each of the information indicating the plurality of symptoms as a part of the health information respectively.

(Appendix 36) A program for causing a computer to execute a storage step of acquiring biological information and storing the biological information in a state of being associated with first time information indicating an acquired date and time, and a receiving step of receiving tag information including health information indicating a certain date and time and second time information indicating the certain date and time from an external input device, wherein the storing step stores the tag information in a state of being associated with the biological information based on the first time information and the second time information indicated by the tag information.

(Addendum 37) The program according to Appendix 36, wherein the storage step retraces for a predetermined period of time the first time information represented by the tag information and stores the tag information in a state associated with the biological information.

(Addendum 38) The program according to Appendix 36, wherein the health information includes information indicating symptoms and body parts, and the storing step stores the biological information for each combination of symptoms and body parts according to the health information indicated by the tag information.

Although the invention made by the inventor has been specifically described based on the embodiment, the present invention is not limited to the embodiment already described, and it is needless to say that various modifications can be made without departing from the gist thereof.

What is claimed is:

1. An input device comprising:
a touch panel configured to:
display a user interface image that displays a body part area for entering body part information;
display the user interface image that displays a symptom area for entering symptom information, after the body part information is entered into the body part area; and
acquire first health information including the body part information from the body part area and the symptom information from the symptom area, and a first time information indicating a first date and time when the first health information is acquired,
a control unit configured to:
receive second biological information and second time information from a biological information collection device that acquires the second biological information in association with the second time information indicating a second date and time when the second biological information is acquired by the biological information collection device;
associate the first health information with the second biological information based on the first time information and the second time information, to generate associated information; and
store the associated information to a storage unit.

2. The input device according to claim 1 further comprising a generation unit configured to generate first tag information based on the first health information and the first time information.

3. The input device according to claim 1 further comprising a notification unit configured to prompt an input of progress information indicating a progress of the first health information.

4. The input device according to claim 1 wherein the touch panel is further configured to:
determine a severity of the symptom according to a distance from a first selected position or a predetermined position in the body part area to a second selected position in the symptom area; and
acquire severity information indicating the severity of the symptom.

5. The input device according to claim 4 wherein the touch panel is further configured to:
display a progress input user interface image that displays a second body part area for entering second body part information;
display the progress input user interface image that displays a second symptom area for entering second symptom information, after the second body part information is entered into the second body part area; and
acquire the progress information including the second body part information from the second body part area and the second symptom information from the second symptom area, and a third time information indicating a third date and time when the progress information is acquired.

6. The input device according to claim 5 wherein the touch panel is further configured to:
determine a changed severity of the symptom according to a distance from a first selected position or a predetermined position in the second body part area to a second selected position in the second symptom area; and
acquire changed severity information indicating the changed severity of the symptom.

7. The input device according to claim 6 wherein the touch panel is further configured to:
display the progress input user interface image that displays a list for entering information on cause or reason of recovery of the symptom; and
acquire cause information including the information on cause or reason of recovery of the symptom from the list.

8. The input device according to claim 7 wherein the touch panel is further configured to:
display the user interface image for entering a plurality of symptoms; and
acquire the first health information including a plurality of body part information from the body part area and a plurality of symptom information from the symptom area, and the first time information indicating a first date and time when the first health information is acquired.

9. The input device according to claim 1,
wherein the control unit is further configured to retrace the first date and time indicated by the first time information for a predetermined time to a retraced time, and
wherein the control unit associates the first health information with the second biological information based on the retraced time and the second time information, to generate associated information.

10. A system comprising:
the input device according to claim 1;
the biological information collection device according to claim 1; and
the storage unit according to claim 1,
wherein the input device further comprises a first communication unit configured to receive the second biological information and the second time information,
wherein the biological information collection device comprises:
a sensor device configured to acquire the second biological information; and
a second communication unit configured to transmit the second biological information and the second time information.

11. An input device comprising:
a display unit;
a display control unit configured to cause the display unit to:
display a user interface image that displays a body part area for entering body part information; and
display the user interface image that displays a symptom area for entering symptom information, after the body part information is entered into the body part area,
an input unit configured to acquire first health information including the body part information from the body part area and the symptom information from the symptom area, and a first time information indicating a first date and time when the first health information is acquired, and
a control unit configured to associate the first health information with a second biological information based on the first time information and a second time information, to generate associated information, and configured to store the associated information to a storage unit, the second biological information being received from a biological information collection device that acquires the second biological information in association with the second time information indicating a second date and time when the second biological information is acquired.

12. The input device according to claim 11 further comprising a generation unit configured to generate first tag information based on the first health information and the first time information.

13. The input device according to claim 11 further comprising a notification unit configured to prompt an input of progress information indicating a progress of the first health information.

14. The input device according to claim 11,
wherein the input unit is further configured to:
determine a severity of the symptom according to a distance from a first selected position or a predetermined position in the body part area to a second selected position in the symptom area; and
acquire severity information indicating the severity of the symptom.

15. The input device according to claim 14,
wherein the display control unit is further configured to cause the display unit to:
display a progress input user interface image that displays a second body part area for entering second body part information;
display the progress input user interface image that displays a second symptom area for entering second symptom information, after the second body part information is entered into the second body part area, and
wherein the input unit is further configured to acquire the progress information including the second body part information from the second body part area and the second symptom information from the second symptom area, and a third time information indicating a third date and time when the progress information is acquired.

16. The input device according to claim 15,
wherein the input unit is further configured to:
determine a changed severity of the symptom according to a distance from a first selected position or a predetermined position in the second body part area to a second selected position in the second symptom area; and
acquire changed severity information indicating the changed severity of the symptom.

17. The input device according to claim 16,
wherein the display control unit is further configured to cause the display unit to display progress input user interface image that displays a list for entering information on cause or reason of recovery of the symptom, and
wherein the input unit is further configured to acquire cause information including the information on cause or reason of recovery of the symptom from the list.

18. The input device according to claim 17,
wherein the display control unit is further configured to cause the display unit to display the user interface image for entering a plurality of symptoms, and
wherein the input unit is further configured to acquire the first health information including a plurality of body part information from the body part area and a plurality of symptom information from the symptom area, and the first time information indicating a first date and time when the first health information is acquired.

19. A biological information collection device comprising:
a sensor device configured to acquire second biological information in association with second time information indicating a second date and time when the biological information is acquired; and
a control unit configured to:
receive first health information and first time information from an input device that acquires the first health information indicating a first date and time when the first health information is acquired;
associate the second biological information with the first health information based on the second time information and the first time information, to generate associated information; and
store the associated information to a storage unit.

20. A system comprising:
the input device according to claim 19;
the biological information collection device according to claim 19; and
the storage unit according to claim 19,
wherein the input device comprises:
a touch panel configured to:
display a user interface image that displays a body part area for entering body part information;
display the user interface image that displays a symptom area for entering symptom information, after the body part information is entered into the body part area; and
acquire the first health information including the body part information from the body part area and the symptom information from the symptom area,
a first communication unit configured to transmit the first health information and the first time information, and
wherein the biological information collection device further comprises a second communication unit configured to receive the first health information and the first time information.
